(19) United States
(12) Patent Application Publication
(10) Pub. No.: US 2017/0072214 A1
(43) Pub. Date: Mar. 16, 2017

(54) Multi-frequency Microwave Acupuncture and Moxibustion Apparatus and its Method for Treating Knee Osteoarthritis

(71) Applicant: Zhongyi Liu, Nanjing (CN)

(72) Inventors: Zhongyi Liu, Nanjing (CN); Jianhua Sun, Nanjing (CN); Yiquan Xu, Nanjing (CN); Zhihui Hu, Nanjing (CN); Qinghua Shao, Nanjing (CN); Lu Chen, Nanjing (CN); Yinghua Lu, Nanjing (CN); Xiang Wu, Nanjing (CN)

(73) Assignee: Zhongyi Liu, Nanjing (CN)

(21) Appl. No.: 15/342,272

(22) Filed: Nov. 3, 2016

Related U.S. Application Data
(63) Continuation-in-part of application No. 13/701,335, filed on Apr. 1, 2014.

(51) Int. Cl. A61N 5/02; A61H 39/08; A61N 5/04

(52) U.S. Cl. CPC A61N 5/025; A61N 5/04; A61H 39/086; A61H 2039/005

(57) ABSTRACT

A multi-frequency microwave acupuncture and moxibustion apparatus characterized in that its structure comprises a microwave oscillator A, a power amplifier A, circulator A, a power divider A, a microwave radiator A, a microwave oscillator B, a power amplifier B, a circulator B, a power divider B, a microwave radiator B, a controller, a display screen, and a power supply unit. Two types of operating frequencies are set: A at 433 MHz, B at 915 MHz, output waveform has continuous wave and four pulse modulation waves respectively at 1 MHz, 5 MHz, 10 MHz and 100 MHz, which are offered to treat different symptoms. Clinical verification shows that the multi-frequency microwave acupuncture and moxibustion apparatus has pretty good curative effects on knee osteoarthritis.

This application claims priority to U.S. patent application Ser. No. 13/701,335 filed 1 Apr. 2014 that is the U.S. national phase of International Application No. PCT/CN2012/000180 Filed on 15 Feb. 2012 which designated the U.S. and claims priority to Chinese Application Nos. CN 201210021439.3 filed on 31 Jan. 2012, the entire contents of each of which are hereby incorporated by reference.

### TECHNICAL FIELD

The present invention belongs to the technical field of medical appliances, and relates to a multi-frequency microwave acupuncture and moxibustion apparatus and the treatment of knee osteoarthritis.

### BACKGROUND ART

Existing technologies in this field have disadvantages. For example, in the patent of application No. 95116466.x, the microwave source is a magnetron operating at 2450 MHz, and the thermal penetration depth is only 1.5 cm, while being high in power consumption and low in efficiency. In the patent of No. ZL93111800.x, although innovative solid state modular is used as medical appliance, limited by technical conditions at that time (1989), the microwave oscillator used is discrete component LC Clapp circuit, and for controller, timer, pulse modulator, and temperature measuring and control devices, analog circuits, mechanical switches, and nixie tubes were used.

Knee osteoarthritis (KOA) is a degeneration disease, of which pathological changes take place in the form of cartilaginous degeneration, synovitis, subchondral bone remodeling, degeneration of the ligament and meniscus, and hypertrophy of the joint capsule. Clinical manifestations include swelling of the knee, stiff knee, and unbearable pains of the knee which gets aggravated at night, disenabling the patient from walking during severe hardships. Such a disease strikes mostly middle aged and elderly people, the morbidity rate among the aged who are older than 60 is 37%-40%, at present, with accelerating aging of population, KOA has become one of the global diseases and impact on daily activities and living quality of middle aged and elderly people, one third of who suffer from disability at last due to few accesses to prompt treatment.

At present, major clinical treatment of knee osteoarthritis comprises the following methods: 1. Western medical therapies including pharmacotherapy, articular injection and debridement arthroscopy, which have fair curative effects while are likely to produce side effects and resistance because of long-term oral administration and infusion of synthetic drugs. Although articular injection and debridement arthroscopy turn out to be good in curative effects, pains of operation trauma are really suffering, the patient could hardly accept it. Knee arthroplasty is the major method of surgery treatment, but it costs quite much and causes serious surgery wounds, possibly leading to certain short-term and long-term complications, and it has problems concerning lifespan, loose and infectivity of the artificial arthroplasty as well as rejection reaction of the body, so this method suits only a small proportion of the KOA patients. 2. Chinese medical therapies: Chinese medicine has its broad and profound science, there have been various kinds of Chinese medical therapies, of which the acupuncture and moxibustion is mostly acknowledged in its curative effects. For example, acupuncture therapy with needles, warm needling moxibustion and small-needle-knife therapy, but these methods may cause slight wounds and hurt, a few people would faint during the acupuncture and moxibustion therapy. Besides, methods such as TDP, oral administration of Chinese medicine, fumigation and external application of Chinese medicine all have certain curative effects. Curative effects of moxibustion shall be acknowledged but it costs the doctor a great deal of time and labor, and the generated smoke would cause environmental pollution.

### SUMMARY OF THE INVENTION

This invention discloses a type of multi-frequency microwave acupuncture and moxibustion apparatus, with the purpose to overcome aforesaid shortcomings of prior art. This apparatus adopts flush type microprocessor, touch type liquid crystal screen, subminiature high efficiency multi-purpose radiator, and solid state modular microwave source (transmitter), so that the subminiature high efficiency multi-purpose radiator (antenna) radiates microwave to relevant acupuncture points of patient, carried to the channels and collaterals, thus dredging the channels and collaterals, regulating qi and the blood, balancing yin yang, and strengthening the body resistance, and eliminating pathogenic factors, so as to effectively cure illness.

Technical solution of this invention: the multi-frequency microwave acupuncture and moxibustion apparatus of this invention comprises a microwave oscillator A, a power amplifier A, a circulator A, a power divider A, a microwave radiator A, a microwave oscillator B, a power amplifier B, a circulator B, a power divider B, a microwave radiator B, a controller, a display screen, and a power supply unit, wherein the microwave oscillator A is connected in series with the power amplifier A, the circulator A, the power divider A, and the microwave radiator A; and the microwave oscillator B is connected in series with the power amplifier B, the circulator B, the power divider B, and the microwave radiator B; a first signal output terminal of the controller is connected to a signal input terminal of the microwave oscillator A, a second signal output terminal of the controller is connected to a signal input terminal of the power amplifier A, a third signal output terminal of the controller is connected to a signal input terminal of the microwave oscillator B, a fourth signal output terminal of the controller is connected to a signal input terminal of the power amplifier B, and a fifth signal output terminal of the controller is connected to a signal input terminal of the display screen; a power input terminal of the controller is connected to a power output terminal of the power supply unit; and a power input terminal of the display screen is connected to a power output terminal of the power supply unit. The microwave radiator A or/and microwave radiator B is/are sucker-type of microwave radiator.

### Advantages of this Invention

1) 2 medical frequencies in the international microwave band L are selected to avoid interference to communication and TV (oscillator A operates at 433 MHz and oscillator B operates at 915 MHz). Thermal penetration is deep and heating homogeneity is good. The solid state modular microwave source used has leakage performance far better than national standard, and good safety performance, high efficiency, and long service life.

2) This apparatus has two operating frequencies. Continuous wave and 4 types of pulse modulated wave (1, 5, 10, 100 Hz) can be selected to treat different symptoms of illness.

3) The subminiature high efficiency radiator is the key component of this apparatus. To adapt to acupuncture points, minimum outline dimensions are: φ25×8 mm for radiator A (5) and φ18×8 mm for radiator B (10). Since operating frequencies are in microwave band L, the difficulty in R&D of subminiature high efficiency radiator is great. Success has only been gained by repeated testing and clinical tests.

4) This invention has dual effect of acupuncture using metal needle and heating of acupuncture points by moxibustion. Good effect is provided for phlogosis, trigeminal neuralgia, and apoplexia and hemi-paralysis etc. Further advantages include absence of trauma, pain, and cross contamination, and easy operation.

5) This apparatus has been tried by specialists and professors in acupuncture and moxibustion of many hospitals. In their opinion, this apparatus has good curative effect, easy operation, and good safety performance, and constitutes an innovation in the medicine of acupuncture and moxibustion.

6) Microwave oscillator used has been changed to voltage controlled oscillator (VCO), which has greatly improved frequency stability and accuracy.

7) Circuits of controller, pulse modulation, timer, and temperature measuring and control etc. adopt new flush type large scale integrated microprocessor, to provide greatly improved detection and control precision.

8) The 10.2-inch color touch type liquid crystal screen used can display various parameters, human body acupuncture point diagram, and acupuncture point formula recommended by WHO to various countries in a straightforward manner.

Operating Principle of this Invention: The microwave source (transmitter) uses subminiature high efficiency radiator (antenna) to radiate microwave to relevant acupuncture points of patient, carried to the channels and collaterals, thus dredging the channels and collaterals, regulating qi and the blood, balancing yin yang, and strengthening the body resistance, and eliminating pathogenic factors, so as to effectively cure illness. In the opinion of Western medicine, the mechanism of microwave treatment is biological thermal effect and biochemical non-thermal effect, through anatomy analysis.

The present invention effectively eliminates existing defects of both Chinese and Western medical therapies for treating knee osteoarthritis, it only requires to paste the radiant surface of radiator onto the body of the patient at positions of point ex-le 4, point st 35, point st 34 and point sp 10, and choose the output waveform at 1 Hz or 5 Hz, output power from 0.25 W to 0.40 W. It may work when the patient feels slightly warm, and it takes 30 minutes each time of treatment, 3 to 5 times a week, with 2 weeks as a course of treatment. The present invention has uniqueness of no wounds, no pains and no pollution, and it is convenient to conduct, with quite good curative effects.

Knee osteoarthritis, also referred to as circumscribed arthritis, proliferative arthritis and degenerative arthritis, is a degeneration disease of which pathological changes take place in the form of cartilaginous degeneration, synovitis, subchondral bone remodeling, degeneration of the ligament and meniscus, and hypertrophy of the joint capsule. Clinical features include pains of the knee, stiff knee, restrictions upon joint mobility and clicking friction sound in joint motion, and it occurs more often after the middle age and more among the female than in the male.

Knee osteoarthritis is one of the common clinical orthopedic diseases and it takes up 40 percent of all types of arthritis, the morbidity rate among the aged who are older than 65 may reach 85%. The aetiological agent of knee osteoarthritis has not been clearly identified so far, possible aetiological agents may include issues concerning gender, age, ethnicity, obesity, heredity and improper exercise intensity. Wherein most aetiological agents may be categorized as primary ones and secondary ones, knee osteoarthritis caused by primary agents mostly occurs among those who are older than, because the body weight exceeds bearing limits of legs, in turn the joint space narrows, the articular cartilage rubs upon each other and leads to damage on the surface of the articular cartilage, with even hyperostosis and osteophyte due to excessive stress loads. While knee osteoarthritis caused by secondary agents varies, it may occur due to any secondary agent of joint damage or diseases, for example, external injuries such as intra-articular fracture, articular capsule damage, cruciate ligament damage, meniscus damage and innominate bone dislocation, furthermore, factors such as articular instability caused by the loose of the knee articular capsule and ligaments around it, varus or valgus deformity caused by various reasons, articular infection, synovial plica syndrome, long-term application of corticoid medicament and autoimmune and metabolic disorders of local segmental articular cartilage can all lead to knee osteoarthritis. According to modern medical science, knee osteoarthritis is a non-inflammatory disease due to degeneration of the knee articular cartilage, with new-born bone at the edge of the joint surface as its feature. Its aetiological agents mainly include mechanical and biological factors, and it has a complicate pathological process of which a number of steps are yet to be revealed.

Currently, both the domestic and international treatment for this disease focuses on symptoms, and the purpose of it is to relieve the pain, alleviate the inflammation, delay the degeneration of cartilage, improve articular functions and avoid or reduce deformities. Clinical treatment has various methods for treating knee osteoarthritis. Western medical therapies include physical treatment, pharmacotherapy (non-steroidal anti-inflammatory drugs and hyaluronic acid, etc.), articular injection, debridement arthroscopy and knee arthroplasty, etc. Chinese medical therapies may quite vary, the internal therapies apply mainly the Chinese medicine for benefiting liver and kidney, strengthening muscles and bones, promoting the circulation of Qi and blood, dispelling the wind and removing dampness, and dispelling the cold and stopping pains, the external therapies include external washing with Chinese medicine, pasting with ointment and fumigation with Chinese medicine. Acupuncture and moxibustion is acknowledged in its curative effects for knee osteoarthritis, methods of which include acupuncture therapy with needles, warm needling moxibustion, fire needling, small-needle-knife therapy, acupoint injection, float needling, and wrist-ankle acupuncture.

Multi-frequency microwave acupuncture and moxibustion apparatus (Model: ZHT-LA), a medical appliance developed with advanced technology, combines acupuncture and moxibustion therapy with modern technologies such as microwave and computer, based on the traditional merdian-collateral theory of China. The operating principle is to radiate through microwave at relative acupoints of pathological tissues to produce warming effects, so as to dredge the meridian, mediate between Qi and blood, balance Yin and Yang, and support healthy energy to eliminate evils, and to cure the disease.

The target acupoints during treatment are point ex-le 4, point st 35, point st 34 and point sp 10, and it takes 30 minutes each time of treatment, 3 to 5 times a week, with 2 weeks as a course of treatment. According to study, multi-frequency microwave acupuncture and moxibustion apparatus can achieve effects of both acupuncture and moxibustion for warming acupoints, its effect of alleviating the pains that KOA patients suffer can be basically equivalent to that of warm needling moxibustion, meanwhile, it has uniqueness of no wound, no pains and no pollution, and it is convenient to operate, which meets comfort level required by patients and proves to be a beneficial supplement to the traditional Chinese acupuncture and moxibustion. It shall get promoted in clinical application.

### BRIEF DESCRIPTION OF THE DRAWINGS

FIG. 1: structural schematic block diagram of multi-frequency microwave acupuncture and moxibustion apparatus.

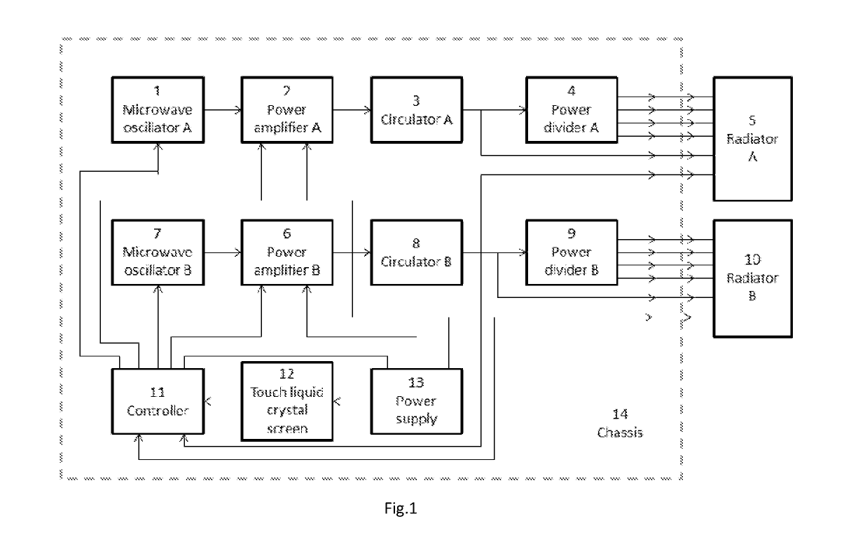

In this FIGURE, 1 is microwave oscillator A, 2 is power amplifier A, 3 is circulator A, 4 is power divider A, 5 is microwave radiator A, 6 is microwave oscillator B, 7 is power amplifier B, 8 is circulator B, 9 is power divider B, 10 is microwave radiator B, 11 is controller, 12 is display screen, 13 is power supply unit, and 14 is chassis.

### DETAILED DESCRIPTION OF THE INVENTION

As shown in the attached FIGURE, the multi-frequency microwave acupuncture and moxibustion apparatus comprises a microwave oscillator A (1), a power amplifier A (2), a circulator A (3), a power divider A (4), a microwave radiator A (5), a microwave oscillator B (6), a power amplifier B (7), a circulator B (8), a power divider B (9), a microwave radiator B (10), a controller (11), a display screen (12), and a power supply unit (13), wherein the microwave oscillator A (1) is connected in series with the power amplifier A (2), the circulator A (3), the power divider A (4), and the microwave radiator A (5); and the microwave oscillator B (6) is connected in series with the power amplifier B (7), the circulator B (8), the power divider B (9), and the microwave radiator B (10); the first signal output terminal of the controller (11) is connected to the signal input terminal of the microwave oscillator A (1), the second signal output terminal of the controller (11) is connected to the signal input terminal of the power amplifier A (2), the third signal output terminal of the controller (11) is connected to the signal input terminal of the microwave oscillator B (6), the fourth signal output terminal of the controller (11) is connected to the signal input terminal of power amplifier B (7), and the fifth signal output terminal of the controller (11) is connected to the signal input terminal of the display screen (12); the power input terminal of the controller (11) is connected to the power output terminal of the power supply unit (13); and the power input terminal of the display screen (12) is connected to the power output terminal of the power supply unit (13).

Shape of microwave radiator A can be: 1) cylindrical (3 types): φ25×8 mm, 4) 31×8 mm, and φ49×14 mm; 2) flat cylindrical: φ67×26 mm; 3) coned: φ100×14 mm; shape of microwave radiator B can be cylindrical (4 types): φ18×8 mm, φ25×8 mm, φ49×14 mm, and φ19×200 mm; and various combinations of such microwave radiator A and microwave radiator B are available to treat different symptoms of illness.

Microwave oscillator A (1) generates continuous wave and 1 Hz/5 Hz/10 Hz/100 Hz pulse modulated waves of operating frequency of 433 MHz for output to power amplifier A (2), where they are amplified, and then output to circulator A (3). This circulator A (3) isolates these waves and sends them to power divider A (4), which divides microwave source power into 4 lines for output to the 4 radiators A (5).

Microwave oscillator B (6) generates continuous wave and 1 Hz/5 Hz/10 Hz/100 Hz pulse modulated waves of operating frequency of 915 MHz for output to power amplifier B (7), where they are amplified, and then output to circulator B (8). This circulator B (8) isolates these waves and sends them to power divider B (9), which divides microwave source power into 4 lines for output to the 4 radiators B (10).

Microwave radiator A (5) and microwave radiator B (10) have various shapes for treatment of different symptoms of illness and multiple acupuncture points.

Operation process of this apparatus is mainly realized by controller (model 9S08DZ60) 11.

Functions of controller (11):

1) Control output waveform of microwave oscillator A (1) and microwave oscillator B (6). Output waveforms include continuous wave, 1 Hz pulse modulated wave, 5 Hz pulse modulated wave, 10 Hz pulse modulated wave, and 100 Hz pulse modulated wave. The pulse modulated waves are most suitable for acupuncture treatment of deep tissue diseases, e.g. prostatitis, periarthritis humeroscapularis, arthritis, and mastitis etc. Select a proper wave for treatment.

2) Set treatment period: When set period expires, an audible sound will be generated and the apparatus will automatically stop.

3) Set control temperature: Treatment temperature is detected in real-time. In case of over-temperature, an alarm will be automatically generated and power output will be cut off.

4) Control and adjust microwave power output.

5) The control display screen (12) displays various settings, treatment parameters, human body acupuncture points diagram, and acupuncture point formula recommended by WHO.

Outline dimensions (mm) of microwave radiators: Microwave radiator A (1) has two shapes: 1) cylindrical: φ25×8 mm (suitable for moxibustion), φ31×8 mm, and φ49×14 mm; 2) flat cylindrical: φ67×26 mm, coned shape: φ100×φ25×14 mm, in total of 6 types. Microwave radiator B (6) has the cylindrical shape: φ18×8 mm, φ25×8 mm, φ49×14 mm, and φ19×200 mm, in total of 4 types. Microwave radiators A and B have a total of 10 types for treatment of different symptoms of illness.

Instance No. 1 of treatment: gonarthritis, with joint inflame, pain, and dropsy:

1. Select operating frequency A (433 MHz); 2. Select 1 Hz or 5 Hz pulse modulated wave; 3. Select φ25×8 radiator A to be placed at acupuncture points.

Place one radiator at each hsiyen point. Place one radiator at the Yangguan point of the knee and one at Ququan point. Use adhesive plaster to fix them tight.

4. Turn on the apparatus and adjust the power till comfortable warmth is felt. 5. Set treatment period to 30 min.

6. Use this treatment once everyday. Normally, after treatment for 3 to 5 times, the pain can be reduced, and subsidence of swelling will be gained. The curative effect is apparent. Instance No. 2 of treatment: paste the radiant surface of radiator onto the body of the patient at positions of point ex-le 4, point st 35, point st 34 and point sp 10, and choose the output waveform at 1 Hz or 5 Hz, output power from 0.25 W to 0.40 W. It may work when the patient feels slightly warm, and it takes 30 minutes each time of treatment, 3 to 5 times a week, with 2 weeks as a course of treatment.

What is claimed is:

1. A multi-frequency microwave acupuncture and moxibustion apparatus, characterized by comprising a microwave oscillator A, a power amplifier A, a circulator A, a power divider A, a microwave radiator A, a microwave oscillator B, a power amplifier B, a circulator B, a power divider B, a microwave radiator B, a controller, a display screen, and a power supply unit, wherein the microwave oscillator A is connected with the power amplifier A, the circulator A, the power divider A and the microwave radiator A in series, while the microwave oscillator B is connected with the power amplifier B, the circulator B, the power divider B and the microwave radiator B; a first signal output terminal of the controller is connected to a signal input terminal of the microwave oscillator A, a second signal output terminal of the controller is connected to a signal input terminal of the power amplifier A, a third signal output terminal of the controller is connected to a signal input terminal of the microwave oscillator B, a fourth signal output terminal of the controller is connected to a signal input terminal of the power amplifier B, and a fifth signal output terminal of the controller is connected to a signal input terminal of the display screen; a power input terminal of the controller is connected to a power output terminal of the power supply unit, and a power input terminal of the display screen is connected to a power output terminal of the power supply unit.

2. The multi-frequency microwave acupuncture and moxibustion apparatus according to claim 1, wherein the microwave radiator A has three shapes: 1) cylindrical: φ25×8 mm (suitable for moxibustion), φ31×8 mm, and φ49×14 mm, in total of three types; 2) flat cylindrical: φ67×26 mm, in total of one type; 3) coned shape: φ100×14 mm, in total one type; microwave radiator B has the cylindrical shape: φ18×8 mm, φ25×8 mm, φ49×14 mm, and φ19×200 mm, in total of four types. The microwave radiators A and B have multiple types for treatment of different symptoms.

3. A method for treating knee osteoarthritis with the multi-frequency microwave acupuncture and moxibustion apparatusof claim 1, characterized in that the multi-frequency microwave acupuncture and moxibustion apparatus comprises a microwave oscillator A, a power amplifier A, a circulator A, a power divider A, a microwave radiator A, a microwave oscillator B, a power amplifier B, a circulator B, a power divider B, a microwave radiator B, a controller, a display screen, and a power supply unit, wherein the microwave oscillator A, the power amplifier A, the circulator A, the power divider A and the microwave radiator A are connected in series, the microwave oscillator B, the power amplifier B, the circulator B, the power divider B and the microwave radiator B are connected in series, the first signal output terminal of the controller is connected to the signal input terminal of the microwave oscillator A, the second signal output terminal of the controller is connected to the signal input terminal of the power amplifier A, the third signal output terminal of the controller is connected to the signal input terminal of the microwave oscillator B, the fourth signal output terminal of the controller is connected to the signal input terminal of the power amplifier B, and the fifth signal output terminal of the controller is connected to the signal input terminal of the display screen; the power input terminal of the controller is connected to the power output terminal of the power supply unit, and the power input terminal of the display screen is connected to the power output terminal of the power supply unit.

4. The method according to claim 3, wherein the temperature of the output pulse modulation wave at 1 Hz and 5 Hz of the multi-frequency microwave acupuncture and moxibustion apparatus is higher than a shell temperature, which is best suitable for moxibustion.

5. The method according to claim 3, wherein solid microwave sources at two frequencies of the multi-frequency microwave acupuncture and moxibustion apparatus both have four-way power output, which is suitable for being applied at 1 to 8 acupoints simultaneously.

6. The method according to claim 3, wherein the radiator A of the multi-frequency microwave acupuncture and moxibustion apparatus has a cylindrical shape, with size of φ25×8 mm, and a standing-wave ratio≦1.13; the radiator B has a cylindrical shape, with size of φ18×8 mm, and a standing-wave ratio≦1.15.

7. The method according to claim 1, wherein the radiator A and the radiator B of the multi-frequency microwave acupuncture and moxibustion apparatus both belong to subminiature efficient microstrip radiators in terms of structure, and the treating method leaves no trauma on body surface.
